(19) United States
(12) Patent Application Publication
(10) Pub. No.: US 2004/0136923 A1
(43) Pub. Date: Jul. 15, 2004

(54) Edible film for relief of cough or symptoms associated with pharyngitis

(76) Inventor: R. Steven Davidson, Woodland Hills, CA (US)

Correspondence Address: PERKINS COIE LLP, POST OFFICE BOX 1208, SEATTLE, WA, 98111-1208, US

(21) Appl. No.: 10/713,544

(22) Filed: Nov. 14, 2003

Related U.S. Application Data
(60) Provisional application No. 60/426,598, filed on Nov. 14, 2002.
(60) Provisional application No. 60/497,186, filed on Aug. 22, 2003.

(51) Int. Cl. A61K 9/68

(52) U.S. Cl. 424/48

(57) ABSTRACT

An edible film comprising an active ingredient for relief of a cough or pharyngitis is disclosed. The edible film comprising a film former and an active ingredient wherein the active ingredient can be selected from active ingredients that have the desired effect of treating cough or pharyngitis. Specific formulations for said film are also disclosed.

### PRIORITY CLAIM

This application claims priority to U.S. Provisional Applications Serial Nos. 60/426,598, filed Nov. 14, 2002 and 60/497,186 filed Aug. 22, 2003, both of which are incorporated herein by reference.

### FIELD OF THE INVENTION

The present invention relates to edible films for relief of cough and/or the symptoms associated with pharyngitis.

### BACKGROUND OF THE INVENTION

Products for the treatment of cough and/or for the treatment of pharyngitis (or “sore throat”) have been known in the art for many years. One popular over-the-counter product for treatment of pharyngitis is a throat spray, where an active ingredient is sprayed into the oral cavity of the user to provide temporary relief of symptoms associated with pharyngitis such as throat pain, irritation, difficulty in swallowing, and hoarseness or laryngitis. Use of a throat spray, however, may be undesirable in public. The spray usually generates a noise drawing unwanted attention to the user. The spray also requires the use of spray mechanisms and containers, which can be relatively expensive when compared to the cost of the product itself and can be inconvenient for use away from the home because of the bulky container and spray mechanism.

Another popular over-the-counter cough suppressant or product for the temporary treatment of pharyngitis is a throat or cough lozenge, where the lozenge slowly dissolves in the oral cavity to provide temporary relief of symptoms associated with pharyngitis and cough. Like throat sprays, lozenges are not always desirable for use in public. While the lozenge does not have the packaging costs of a spray or the inconvenience of carrying a bulky spray mechanism for use in public, lozenges often take an extended period of time to dissolve forcing the user to “suck” on the lozenge for an extended period which is not always socially acceptable and does not provide a quick burst of immediate relief from cough or the symptoms associate with pharyngitis. Lozenges can also be comprised of a candy filler material, as describe in U.S. Pat. No. 5,055,461 to Keller et al., providing extra, unnecessary calories that may be undesirable for the user.

Often the user would prefer inexpensive, convenient and immediate relief from the symptoms associated with pharyngitis and cough without having to carry bulky items away from the home or without having to suck on a candy for an extended period.

In addition, the active ingredient in products for temporary relief of symptoms associated with pharyngitis and cough are often active pharmaceuticals. Users, however, occasionally desire an alternate means to relieve cough and pharyngitis that does not comprise active pharmaceuticals and instead uses natural ingredients to provide similar relief.

Finally, edible thin films are well known in the art. An example of such a strip is described in U.S. Pat. No. 6,419,903 to XU et al. This type of strip is designed to rapidly dissolve in the oral cavity, typically to deliver a breath freshening agent or other oral care product to the user. Such products may have other applications.

Because of the foregoing reasons there is a desire in the field for alternate methods to deliver an active ingredient to relieve cough and/or the symptoms associated with pharyngitis that are less costly and more convenient than current methods. There is also a desire for alternate formulations of the active ingredients for the relief cough and/or the symptoms associated with pharyngitis.

### SUMMARY OF THE INVENTION

It is an object of the present invention to provide an alternate method for the delivery of an active ingredient for the relief of a cough and/or the symptoms associated with pharyngitis.

It is another object of the present invention to provide alternate formulations of the active ingredients for the relief of a cough and/or the symptoms associated with pharyngitis.

These and other aspects of the present invention which may become obvious to those skilled in the art through the following description of the invention are achieved by an edible film for delivery of an active ingredient to or via the oral cavity.

In one embodiment of the invention, an edible film according to the present invention is disclosed comprising an active ingredient wherein said active ingredient comprising a mixture of essential oils and/or natural ingredients for the treatment of a cough or the symptoms associated with pharyngitis.

In a second embodiment of the invention, an edible film according to present invention is disclosed comprising an active pharmaceutical for the treatment of a cough or the symptoms associated with pharyngitis.

In a third embodiment of the invention, a method according to the present invention is disclosed comprising an edible film having an active ingredient and placing the edible film into the oral cavity such that the film dissolves thereby delivering the active ingredient to the oral cavity.

### DETAILED DESCRIPTION OF THE PREFERRED EMBODIMENTS

In order to fully understand the manner in which the above-recited details and other advantages and objects according to the invention are obtained, a more detailed description of the invention will be rendered by reference to specific embodiments thereof.

An edible film to ameliorate a cough and/or pharyngitis according to the present invention is described having an edible film as a carrier and an active ingredient or medicant carried by the carrier wherein the film dissolves in the oral cavity of a user thereby delivering an appropriate dosage of the active ingredient to the user.

The appropriate edible film carrier can be selected by one of ordinary skill in the art depending upon factors including the desired rate of dissolution, desired oral feel for the user, the compatibility of the thin film carrier and the active ingredients, production constraints, costs, or other factors. The film can also be thick or thin depending upon these same factors.

The desired rate for dissolution can vary depending of the specific application for the edible film. For example, for immediate delivery of the active ingredient, the film can be manufactured to rapidly dissolve in the oral cavity thus delivering the entire dosage of active ingredient at one time. The film can also be manufactured to dissolve over an extended period regulating the amount of active material delivered to the oral cavity over a desired length of time.

Specific film formulations and methods of manufacture are known in the art, for example see U.S. Pat. No. 5,948,430 to ZERBE et al., incorporated herein by reference. Each film formulation usually comprises film formers, bulking agents, softeners, intense artificial sweeteners, sugar alcohol, natural sweeteners, flavors, cooling agents, surfactants, coloring agents, oils, and drying agents. These ingredients are well known and widely available in the food industry.

The primary ingredient for an edible film according to the present invention is the film former, which in most cases can be any water soluble film former. Film formers include but are not limited to pullulan, guar gum, pectin, xanthan gum, alginates, gelatin, starches (including corn, potato, rice or tapioca), modified starches, matltodextrins, wheat gluten, carboxymethylcellulose, carrageenan konjac or locust bean gum.

The active ingredient can be any active pharmaceutical. Such ingredients for the treatment of pharyngitis include but are not limited to menthol, phenol or benzocaine. Naturally occurring herbs, plants, vitamins and oils can also relieve symptoms of pharyngitis and cough and therefore can also be used as the active ingredients in the edible film. Natural ingredients for the treatment of pharyngitis and cough include but are not limited to the ingredients listed in Table 1. Specific formulations of said ingredients may be selected by one of ordinary skill in the art depending on the specific application and other factors such as the desired effect or flavor of the edible film.

TABLE 1
Natural Ingredients For The Treatment Of Pharyngitis And Cough

| | Ingredient (Botanical name*) |
|---|---|
| Herbs | Adrogrophis paniculata |
| | Agrimony (agriminio eupatoria) |
| | bistort (polygonum bistora) |
| | blue gum tree (eucalytus globulus) |
| | club moss (lycopodium clavatum) |
| | fenugreek |
| | garden thyme (thymus vulgaris) |
| | ginger |
| | golden seal (hydrastid candenis) |
| | kava kava |
| | lady's mantle (alchemilla vulgaris) |
| | lavender (lavedula spp.) |
| | lobelia |
| | loosestrife (lythrum salicaria) |
| | Marsh cudweed (gnophthalum uliginosum) |
| | myrrh (commiphora molmol) |
| | peppermint (mentha piperita) |
| | phosphorous |
| | poker root (phytolacca americana) |
| | pokeweed (phytolacca decandra) |
| | purple cone flower (echinacea puprea) |
| | purple sage (salvia officenalis) |
| | S. Benzoin, gum Benjamin |
| | solanum |
| | tea tree oil (melaeuca alternifolia) |
| | wild indigo (baptisma tinctoria) |
| Tree and Plant | aloe |
| sources | bee pollen |
| | blackberry |
| | camphor oil |
| | cayenne |
| | elderberry |
| | gum arabic |
| | honey |
| | licorice extract |
| | maitake extract |
| | olive leaf extract |
| | sage oils |
| | sarsparilla |
| | sweet oil of birch |
| | shitake extract |
| | slippery elm |
| | willow bark |
| Vitamins and | co-enzyme Q10 |
| minerals | collodial silver |
| | vitamin C |
| | vitamin E |
| | zinc |
| Bacteria | lactobacillus acidophilus |
| Essential oils and | cinnamon oil |
| flavors | clove oil |
| | fennel seed oil |
| | lemon oil |
| | menthol eucalyptus oil |
| | peppermint oil |
| | rosemary oil |
| | spearmint oil |
| | wild cherry oil |

*if available

### EXAMPLE I

An edible film according to the present invention is described comprising a bi-layer film. The film consists of one water soluble layer that serves as a substrate layer or active layer and a second dry coat layer. The second dry coat layer settles into the substrate layer affixing itself to that bottom layer. While active ingredients may be contained in either layer, preferably the second dry coat layer will contain one or more active ingredients such as menthol or benzocaine or both. The dry coat layer is applied to the thin film surface after partial curing of the first (bottom) layer, affixing itself to this bottom layer. Said dry coat layer and similar layers are especially effective with low dose active ingredients that require a very low moisture environment to remain stable. The second layer can also contain substrates and partitioning agents.

The film is of a size such that it is fast dissolving. The weight per strip may vary. Said weight of the strip may be in the ranges of about 10 to 80 mg, about 20 to 70 mg, about 30 to 60 mg and about 50 mg. The maximum dosing per strip may also vary depending on the choice of active ingredient. Said maximum dosing is preferably 12.5 mg. Active ingredients can be delivered in a solid or liquid format and depending on dose levels, the Active ingredients can be oil or water soluble. Active ingredients that are stable in aqueous systems are preferred. Active ingredients that are not stable in an aqueous system, however, though not preferred, may still be used. Preferably, the dosage per serving is 1-2 strips but may vary depending on the size of the individual strip and other factors known one skilled in the art.

Individual strips can be made in virtually any size, preferably the strips are 13/16 inch by 1 ¼ inch rectangles. The thickness of the first layer is preferably in between about 0.040 to 1.1 micrometers. The thickness of the second-dry coat preferably in the range of about 0.007 to 0.02 micrometers. The thickness of the particularly layers may be more or less than the values recited herein depending on known to one skilled in the art such as load and processing challenges.

Table 2 lists a formulation for a strip according to the present invention.

TABLE 2
Edible Film Formulation*

| Ingredient | Preferred wt % | More preferred wt % |
|---|---|---|
| Water | 0 to 25 | 5 to 15 |
| N&A Cherry Oil | 0 to 25 | 10 to 20 |
| Carrageen | 0 to 10 | 3 to 6 |
| Acsulfame | 0 to 0.1 | 0.2 to 0.6 |
| Potassium | | |
| Sucralose | 0 to 5 | 1 to 3 |
| Lecithin | 0 to 1 | .2 to 0.6 |
| Benzocaine | 0 to 12 | 3 to 9 |
| Pectin** | 20 to 60 | 35 to 50 |
| Glycerin | 0 to 10 | 2 to 8 |
| Sodium Benzoate | 0 to 2 | .05 to .2 |
| Poly Sorbate 80 | 0 to 0.5 | .05 to .35 |
| Menthol | 1 to 12 | 3 to 9 |
| Carboxylmethyl | 1 to 12 | 3 to 9 |
| Cellulose | | |

*finished film contains 8 to 10% moisture by weight
**Pectin may be replaced by up to 5% of one of the following: Gelatin, Maltodextrin, Modified Food Starch, TiO2, and Acacia Gum.

Said formulation will deliver approximately 3 mg of menthol and 3 mg of benzocaine per dose. Further, it may be advantage to include 15 to 20% active ingredient average as needed to complete two year stability for the strip.

### EXAMPLE II

Table 3 lists a specific formulation for an edible film according to the present invention.

TABLE 3
Edible Film Formulation*

| | Preferred | More | Most |
|---|---|---|---|
| Ingredient | wt % | preferred wt % | preferred wt % |
| Tapioca Starch | 2 to 65 | 18 to 25 | 22.8 |
| Pullulan | 3 to 85 | 15 to 25 | 20 |
| Pectin | 1 to 30 | 15 to 25 | 20 |
| Gum Arabic | 0.05 to 8 | 2 to 4 | 3 |
| Maltodextrin | 2.5 to 15 | 4 to 6 | 5 |
| Polysorbate | 0.01 to 2 | 0.075 to 0.175 | 0.15 |
| Sodium | 0.05 to .75 | 0.1 to 0.4 | 0.25 |
| Saccharin | | | |
| Alginate | 5 to 30 | 8 to 12 | 10 |
| Carrageenan | 1 to 5 | 1.5 to 3 | 2.5 |
| Clove Oil | 0.25 to 10 | 2 to 7 | 5 |
| Cinnamon Oil | 0.25 to 10 | 2 to 7 | 5 |
| Echinacea | 1 to 10 | 1 to 3 | 2.5 |
| Vitamin E | 0.25 to 5 | 0.5 to 2 | 1 |
| Slippery Elm | 1 to 10 | 2 to 6 | 5 |
| Aloe Vera | 1 to 7.5 | 1.5 to 3.5 | 2 |

*wt % is dry weight (finished film contains 8 to 10% moisture by weight)

### EXAMPLE III

For the edible film of example one, a clinical laboratory study was conducted to evaluate the effectiveness of the edible film in producing a numbing anesthetic effect in normal individuals. The study included 26 subjects of which three were males and tewnty three were females. The ages of the subjects ranged from 23 to 71 years. All 26 subjects completed th study.

To be included in the study all subjects must have met the following criteria: age 18 or over, must not be using a topical anesthetic product, considered normal (i.e., with no throat pain or visable signs of sore throat), must not be presently taking antibiotics or have taken antibiotics at any time 4 weeks prior to the study, must complete a medical history form with the understanding and signing of an informed consent form, and females must not be pregnant or nursing.

The test material included 0.01 Batch #2: with actives, and 0.02 Batch #1: without actives.

The testing lasted two days. During testing the subjects were instructed not to eat or drink coffee or tea for at least two hours prior to evaluating the edible film, not to perform any type of oral hygene, not to chew gum and not to smoke.

On test day one each subject was given one edible fim to evaluate for numbing effects. Each subject was instructed to 1) remove one strip from the dipenser and place on the back of the subjects tongue to dissolve in the mouth. The subjects were then instucted to use a second strip immediately after the first one dissolves and one minute after the second strip dissolves, complete the evaluation form.

On test day two all subjects repeated the evaluation as previously described using the an edible film from a different batch than used on the first day.

Subjects were instructed to notify the clinical laboratory immediately of any adverse reactions to the product. Product usage would be discontinued and an evaluation would be conducted by a trained technician. If deemed necessary, there would be a referral for medical intervention. No adverse experiences were reported at any time during the course of the study.

The t-test (Dependent) was used to determine the significance of any differences between the subject-percieved numbing measurements for each edible film.

Table 4 summaraizes the statistical analysis of the subject perceived numbness measurements. There was a highly significant, subject-perceived feeling of numbness between the active and non-active strips.

Table 5 shows subject demographics and test product randomization.

In summary, under the conditions of the study, test material, Batch #2: with actives induced a highly significant, subject-perceived feeling of numbness when compared to test material, Batch #1: without actives.

TABLE 4
Comparative Subject-Perceived “Numbness”
(100 mm open linear scale)

| | Batch #1: without actives | Batch #2: with actives |
|---|---|---|
| Subject # | (mm)* | (mm) |
| 1 | 0 | 81 |
| 2 | 5 | 84 |
| 3 | 0 | 20 |
| 4 | 5 | 85 |
| 5 | 0 | 80 |
| 6 | 0 | 30 |
| 7 | 0 | 40 |
| 8 | 0 | 90 |
| 9 | 16 | 86 |
| 10 | 3 | 96 |
| 11 | 2 | 86 |
| 12 | 6 | 50 |
| 13 | 0 | 40 |
| 14 | 20 | 80 |
| 15 | 2 | 41 |
| 16 | 2 | 81 |
| 17 | 10 | 80 |
| 18 | 10 | 90 |
| 19 | 0 | 80 |
| 20 | 11 | 65 |
| 21 | 1 | 100 |
| 22 | 0 | 40 |
| 23 | 0 | 40 |
| 24 | 0 | 46 |
| 25 | 10 | 80 |
| 26 | 0 | 70 |
| Mean | 4.0 | 67.7 |

*0 = no perceive numbness
100 = complete perceived numbness
t-Test (Dependent)
t 14.898
df 25
One-tailed p < 0.000
Two-tailed p < 0.0000
r 0.360 (Highly Significant)

TABLE 5
SUBJECT DEMOGRAPHICS AND RANDOMIZATION

| Subject # | Initials | Age | Sex | Study Day 1 | Study Day 2 |
|---|---|---|---|---|---|
| 1 | LB | 56 | F | .01 Batch # 2 | .02 Batch # 1 |
| 2 | JE | 58 | F | .02 Batch # 1 | .01 Batch # 2 |
| 3 | RB | 30 | M | .01 Batch # 2 | .02 Batch # 1 |
| 4 | BK | 68 | F | .02 Batch # 1 | .01 Batch # 2 |
| 5 | CG | 71 | F | .01 Batch # 2 | .02 Batch # 1 |
| 6 | LA | 27 | F | .02 Batch # 1 | .01 Batch # 2 |
| 7 | AF | 28 | F | .01 Batch # 2 | .02 Batch # 1 |
| 8 | LI | 35 | F | .02 Batch # 1 | .01 Batch # 2 |
| 9 | MS | 29 | F | .01 Batch # 2 | .02 Batch # 1 |
| 10 | PS | 46 | F | .02 Batch # 1 | .01 Batch # 2 |
| 11 | LD | 29 | F | .01 Batch # 2 | .02 Batch # 1 |
| 12 | LE | 47 | F | .02 Batch # 1 | .01 Batch # 2 |
| 13 | KE | 23 | F | .01 Batch # 2 | .02 Batch # 1 |
| 14 | DC | 41 | F | .02 Batch # 1 | .01 Batch # 2 |
| 15 | JV | 28 | M | .01 Batch # 2 | .02 Batch # 1 |
| 16 | HG | 65 | F | .02 Batch # 1 | .01 Batch # 2 |
| 17 | KS | 64 | F | .01 Batch # 2 | .02 Batch # 1 |
| 18 | MB | 27 | F | .02 Batch # 1 | .01 Batch # 2 |
| 19 | RP | 36 | F | .01 Batch # 2 | .02 Batch # 1 |
| 20 | AT | 58 | F | .02 Batch # 1 | .01 Batch # 2 |
| 21 | ND | 39 | F | .01 Batch # 2 | .02 Batch # 1 |
| 22 | LA | 47 | F | .02 Batch # 1 | .01 Batch # 2 |
| 23 | NR | 41 | F | .01 Batch # 2 | .02 Batch # 1 |
| 24 | LE | 31 | F | .02 Batch # 1 | .01 Batch # 2 |
| 25 | AR | 30 | M | .01 Batch # 2 | .02 Batch # 1 |
| 26 | CM | 45 | F | .02 Batch # 1 | .01 Batch # 2 |

Any standard manufacturing procedure known in the art may be used to manufacture the film. An example of such a process can be found in U.S. Pat. No. 5,948,430 to ZERBE et al.

Further to the production method described in U.S. Pat. No. 5,948,430 to ZERBE et al., the production of an edible film according to the present invention can also include an aeration step. This step includes aerating the mass prior to application onto a substrate. Aeration is most preferably achieved through mechanical agitation, mechanical reaction, or carbon dioxide aeration. The aeration step produces an edible film having greater thickness and lower density than without aeration.

A further embodiment of the present invention includes an improved film and method for making the same. The film can be used on living cells. Formation of the medicant-containing layer in the film does not require a solvent and minimizes the likelihood of damage from heat and shear. The rate of dissolution or delivery of the medicant by the film can be readily adjusted. The medicant-containing layer, while minimizing the likelihood of heat induced medicant damage, permits heat to be utilized to form a coating on the edible film. Hydrophilic components can be readily incorporated in larger concentrations during production of the medicantcontaining layer.

Further, the present invention includes an improved composition for delivering a medicant in the oral cavity. The composition includes an applied coating and a film layer.

The film layer is made from any polymer, softener, filler, matrix, or other composition. The film has an acceptable dissolution rate in the oral cavity for a particular thickness of film. For example, if the film has a thickness of 50 microns, it may be desirable for the film to dissolve in the oral cavity within about fifteen seconds. Or it may be desirable for the film to dissolve more slowly. By way of example, and not limitation, the film can be made with pullulan, modified starch, pectin, carageenan, a maltrodextrin, or alginate.

The applied coating is a powder matrix including one or more medicants. The medicant can be contained in a powder carrier, or can itself be a powder. One advantage of the powder matrix is that it ordinarily does not require the use of a solvent. Another advantage of the powder matrix is that it ordinarily can, if desired, include in addition to the medicant a variety of different auxiliary compositions. A further advantage of the powder matrix is that it can be admixed in a fluidized bed that minimizes the generation of shear and heat. In a fluidized bed dry air or another gas is dispersed upwardly through a plurality of openings to suspend and intermix particulate. Any desired means can be used to admix powders. Another advantage of mixing or suspending powder in a fluidized bed is that the dry air suspending the powder particles tends to prevent agglomeration of the particles. The admixed powder matrix can also be stored (i.e., suspended) in the fluidized bed, prior to the application of the admixed powder matrix to the film layer. The powder matrix can be applied in any desired manner, including sifting, screening, atomization, static, mechanical agitation, etc. For example, the powder matrix can be atomized through a Nordson or similar static spray gun using compressed air. One such gun creates a fine mist spray of powder particles. The gun statically electrically charges the powder particles so they adhere to a surface of the film layer that is receiving the powder particles. Another process for applying the powder particles is to admix the particles with a liquid carrier to form a particle-liquid solution. The particle-liquid solution is sprayed on the film layer. The liquid carrier evaporates, leaving the powder particles on the film. The liquid carrier preferably does not cause the powder particles to dissolve in the liquid carrier.

One auxiliary composition that can be included in the powder matrix with the medicant is a composition that dissolves slowly over a selected period of time. Such an auxiliary dissolution control composition can be utilized to slow the release of medicant in the oral cavity. Examples of this kind of auxiliary composition are, without limitation, gel forming compositions like carrageenan, gelatin, alignates, pullulan, PVP, and other hydrophilic materials; cyclodextrin; and, inert materials like calcium and fibers. For example, the fibers can comprise carboxymethylcellulose.

Another auxiliary composition the can be included in the powder matrix with the medicant is an absorption composition that absorbs water or saliva. Such an auxiliary absorption composition can be also be used to slow the release of medicant, and/or, to form a gel. The gel can, if desired, cause the strip to become chewable, similar to a very soft jelly-bean. As used herein, an auxiliary composition is termed a gel if, when it is placed in the oral cavity or in contact with another source of bodily liquid, (1) the auxiliary composition absorbs at least four times it weight of water or of saliva or other aqueous solution in a selected period of time, or (2) the auxiliary composition swells to at least three times its thickness in a selected period of time. The selected period of time can vary but preferably is from five seconds to fifteen minutes, most preferably five seconds to five minutes. Examples of gel auxiliary compositions include, without limitation, carboxymethylcellulose, pectin, modified starches, gelatin, and carrageenan. These compositions can be used alone or in combination, One advantage of a gel is that it tends to slow the dissolution of the medicant and to maintain the medicant in the oral cavity for a longer period of time.

A further auxiliary composition that can be included in the powder matrix is a composition that, when placed in the oral cavity in contact with the mucosa therein, adheres to the mucosa. The concentration of such auxiliary adhesion compositions in the powder matrix can be adjusted to vary the length of time that the film adheres to the mucosa or to vary the adhesive forces generated between the film and mucosa. The auxiliary adhesion compositions adhere to the oral mucosa or to mucosa or tissue in other parts of the body, including the mouth, nose, eyes, vagina, and rectum. Examples of auxiliary adhesion compositions include carboxymethycellulose, polyvinyl alcohol, polyvinyl pyrrolidone (povidone), sodiumalginate, methyl cellulose, hydroxyl propyl cellulose, hydroxypropylmethyl cellulose, polyethylene glycols, carbopol, polycarbophil, carboxyvinyl copolymers, propylene glycol alginate, alginic acid, methyl methacrylate copolymers, tragacanth gum, guar gum, karaya gum, ethylene vinyl cetate, dimenthylpolysiloxanes, polyoxyalkylene block copolymers, and hydroxyethylmethacrylate copolymers. All examples of composition provided herein are given without limiting the use or inclusion of other comparable or functionally equivalent compositions even though such comparable or functionally equivalent compositions are not listed.

Still another auxiliary composition that can be included in the powder matrix is a flow composition that, when subjected to a curing process, flows to form a smoother or shinier coating on the exterior of the film layer. One preferred curing process is heating the film layer with powder coating to a selected temperature above 76 degrees F. to cause the auxiliary flow composition to soften and flow. Examples of this kind of auxiliary composition are lipids (including various animal and vegetable fats) waxes, particularly low melting point waxes, and polyols, particularly low melting point polyols that can be admixed in powder form or than can included be in powder particles containing a medicant or other compositions. The medicant itself, may also have the property of flowing at an elevated temperature in excess of 76 degrees F. to form a smoother or shinier coating.

Other auxiliary compositions that can be included in the powder matrix include, without limitation, bulking agents, fillers, pigments (coloring), flavorings, and sweeteners.

Combinations of auxiliary compositions can be included in the powder matrix to achieve a desired function. For example, if it is desired to slow the dissolution of a medicant, less soluble fillers and fibers can be included in the powder matrix along with a high concentration of polymers that have a very high degree of ability to adhere to the oral mucosa lining the mouth.

The powder matrix is normally administered to the film layer to form the applied coating after the film layer has been manufactured.

The dry powder matrix will normally contain a minor amount of retained or bound water or other liquid, typically less than about ten percent by weight. The level of moisture in the powder matrix normally should not cause the powder particles to stick or adhere to one another during intermixing of powders to form the powder matrix and during application of the powder matrix to the film layer.

By way of example, and not limitation, the film layer can be produced using a highly water-soluble polymer comprising a natural or synthetic water-soluble polymer. The polymer preferably has good film moldability, produces a soft flexible film, and is safe for human consumption. One such polymer can be a water-soluble cellulose derivative like hydroxypropyl cellulose (HPC), methyl cellulose, hydroxypropyl alkylcellulose, carboxymethyl cellulose or the salt of carboxymethyl cellulose. Or, the polymer can comprise an acrylic acid copolymer or its sodium, potassium or ammonium salt. The acrylic acid copolymer or its salt can be combined with methacrylic acid, styrene or vinyl type of ether as a comonomer, poly vinyl alcohol, poly vinyl pyrrolidone, polyalkylene blycol, hydroxy propyl starch, alginic acid or its salt, poly-saccharide or its derivatives such as trangacanth, bum gelatin, collagen, denatured gelatin, and collagen treated with succinic acid or anhydrous phthalic acid. By way of example, the following can be included in the powder matrix as adhesives: poorly water-soluble cellulose derivatives including ethyl cellulose, cellulose acetate and butyl cellulose; shellac; higher fatty acids including steric acid and palmitic acid. The following can also, without limitation, be used to produce the film layer: pullulan, maltrodextrin, pectin, alginates, carrageenan, guar gum, other gelatins, etc.

Bulking agents that can be included in the powder matrix include, by way of example and not limitation, avicel, sugar alchohols including manitol and sorbitol and xylitol and isomalt, lactic sugar, sorbitol dextrin, starch, anhydrous calcium phosphate, calcium carbonate, magnesium trisilicate, silica, and amylase.

The size of particulate in the powder matrix can vary as desired, but is preferably in the range of 10 mesh to 400 mesh or finer, preferably 40 mesh to 300 mesh.

The thickness of the film layer can vary as desired, but typically is in the range of 0.01 mm to 3.00 mm, preferably 0.03 mm to 1.00 mm.

The powder matrix can be applied to one or both sides of the film layer. The film layer includes upper outer surface on the top of the film layer and includes a lower outer surface on the bottom of the film. The upper outer surface is generally parallel to the lower outer surface. The top of the film is generally parallel to the bottom of the film. The thickness of the powder matrix layer can vary as desired, but is preferably in the range of 0.001 mm to 3.00 mm, preferably 0.01 mm to 1.00 mm.

If desired, after the powder matrix layer is applied to the film layer, an additional layer or layers can be applied over the powder matrix layer to seal the powder matrix layer, slow the dissolution of the medicant from the powder matrix layer, etc.

If desired, multiple powder matrix layers can be applied to the film layer. The film layer can comprise a laminate of two or more layers. Methods for producing the film layer and incorporating plasticizers, bulking agents, taste modifying agents, pigments, etc. in the film layer are well known in the art and not described in detail herein. Since the medicant is being applied to the film layer in a dry powder form, the likelihood of adverse interactions between the medicant and compositions comprising the film layer is lessened.

Unless otherwise specified or required by the context, the term edible as used herein is used interchangeably with the term orally consumable, and generally means that the article may be placed in the mouth, oral cavity, on the tongue, or the like, without significant detrimental effect to the recipient.

In certain embodiments the compositions and films of the present invention may contain at least one flavoring and/or odorant composition that renders the composition or film palatable. Any effective flavor or odor may be used. The flavoring or odor agent or agents are present in any effective amount, including, for example, in an amount ranging from about 0.5 to 40 wt. %, 1 to 30 wt. %, 5 to 15 wt. %, 0.5 to 15 wt. %. The flavorings may be natural or artificial, or combinations thereof.

In certain embodiments the compositions and films of the present invention may contain at least one ingredient or agent that is pharmaceutically active. Any effective pharmaceutically active ingredient or agent may be used in accordance with the present invention. The pharmaceutically active ingredient or agent may be present in any effective amount, including, for example, in an amount ranging from about 0.5 to 40 wt. %, 1 to 30 wt. %, 5 to 15 wt. %, 0.5 to 15 wt. %.

Unless otherwise specified or required by the context, the edible films of the present invention may be manufactured in any effective manner. U.S. patent application Ser. Nos. 20010022964, 20020131990 and 20020019447 and U.S. Pat. Nos. 6,419,903, 3,931,146, 5,411,945, 6,010,716, 5,629,003, 5,948,430, 6,177,096, 6,284,264, 5,700,478, 6,449,925, 4,072,551, 4,083,741, all of which are incorporated herein by reference as if fully set forth herein, describe methods for making edible films. These, and other methods known in the art, or described herein, may be used in accordance with the present invention.

### EXAMPLE IV

3.4 g of hydropropyl cellulose and 0.4 ml of macrogol-400 (polyethylene glycol) are dissolved in 60 g of ethyl alcohol to produce a cellulose-alcohol solution. Nine milliliters of distilled water containing 90 mg of dissolved predonisolone is added to the cellulose-alcohol solution to produce a film forming composition. The film forming composition is poured into a film molding frame placed on a teflon plate. The area of teflon plate circumscribed by the frame is 9.5 square centimeters. The film forming composition is dried to form a film layer. The film layer includes an upper outer surface on top of the film layer and includes a lower outer surface on the bottom of the film layer. The lower outer surface is generally parallel to the upper outer surface. The film layer has a thickness of 40 microns. As noted, any desired prior art process and/or materials can be utilized to produce the film layer.

Benzocaine powder (as a medicant) is combined with carboxymethylcellulose powder (as an adhesive), modified food starch (as a bulking agent), carrageenan (as adhesive), sucralose (intense sweetener), talc (as flow/partitioning agent), and menthol (as a medicant) in a fluidized bed container to form a powder matrix. The resulting powder matrix includes 3.76% by weight of benzocaine powder, 2.6% by weight percent of carboxymethylcellulose powder, 85.43% by weight of modified food starch, 3.76% by weight menthol, 2% by weight carrageenan, 0.45% by weight sucralose, and 2.0% by weight magnesium trisilicate (talc). The powder matrix is drawn from the fluidized bed container and is applied to the upper exposed surface of the film layer to a substantially uniform thickness of 60 microns. The powder matrix is atomized through a Nordson or similar static spray gun using compressed air. See, for example Nordson Corporation's KINETIC (TM) spray systems (www.nordson.com). The gun creates a fine mist spray of powder particles. The gun statically electrically charges the powder particles so they adhere to the upper surface of the film layer. If desired the powder matrix can also be applied to the lower or bottom surface of the film layer. The powder matrix layer and film layer together comprise a medicant composition. The medicant composition can be applied to mucous membrane at various areas of the body.

### EXAMPLE V

A film layer is prepared as follows. Xanthan gum (1.5% by weight), locust bean gum (1.5% by weight), carrageenan (1% by weight) and pullulan (9.5% by weight) are mixed and hydrated in hot purified water (86.5% by weight) to form a gel. The gel is stored in a refrigerator overnight at a temperature of approximately four degrees C to form a film layer. The film layer has a thickness of 55 microns.

Coral calcium powder (as a medicant) is combined with carboxymethylcellulose powder (as an adhesive), modified food starch (as a bulking agent), carrageenan (as adhesive), sucralose (intense sweetener), talc (as flow/partitioning agent), menthol (as a medicant), and a lipid in a fluidized-bed container to produce a powder matrix. The lipid is BENEFAT™. BENEFAT is used by DANISCO to designate salatrim, which is the abbreviation for long and short chain triglyceride molecules. The resulting powder matrix includes 3.76% by weight of coral calcium powder, 2.6% by weight percent of carboxymethylcellulose powder, 73.43% by weight of modified food starch, 3.76% by weight menthol, 2% by weight carrageenan, 0.45% by weight sucralose, 2.0% by weight magnesium trisilicate, and 12% by weight of the lipid. The lipid preferably is in powder form. If the lipid initially is in liquid form, it can be plated on a particulate absorbent to produce a flowable powder. The particulate absorbent could, for example, be talc.

The powder matrix is drawn from the fluidized bed container and is applied to the upper exposed surface of the film layer to a uniform thickness of 150 microns. The powder matrix is atomized through a Nordson or similar static spray gun using compressed air. The powder matrix layer and film layer together comprise a medicant composition.

Ideally, the melting point of the lipid is close to temperature at which the film layer is dried. For example, the film layer (along with the powder matrix layer applied to the film layer) is typically dried at about 200 degrees F. The lipid preferably has a softening point or melting temperature of about 200 degrees F. so that the temperature at which the film layer is dried is the ideal softening point for the lipid. If the melting temperature of the lipid is too low in comparison to the temperature at which the film layer is dried, the lipid can melt and run off the film.

The medicant composition is cured using any desired heat treatment process. The presently preferred process comprises a first step during which the medicant composition is heated by a microwave or infrared transmitter. The time spent by the medicant composition under the transmitter varies depending on the amount of moisture to be removed, but typically is fifteen to twenty seconds. The microwave/infrared bombardment facilitates proper heating of the film layer by generating heat in the film layer. During the second step of the heat treatment process the medicant composition is heated to 200 degrees F. in a convection oven for a desired length of time to dry the medicant composition. The length of time the medicant composition is in the convection oven can vary but is typically presently about three to four minutes. During the foregoing heat treatment process, the lipid powder particles soften and flow to produce a smoother powder matrix layer on the film layer. The smoother powder matrix layer also improves the feel to an individual of the medicant composition in the mouth because the medicant composition is not as dry on the tongue.

### EXAMPLE VI

3.4 g of hydropropyl cellulose and 0.4 ml of macrogol-400 (polyethylene glycol) are dissolved in 60 g of ethyl alcohol to produce a cellulose-alcohol solution. Nine milliliters of distilled water containing 90 mg of dissolved predonisolone is added to the cellulose-alcohol solution to produce a film forming composition. The film forming composition is poured into a film molding frame placed on a teflon plate. The area of teflon plate circumscribed by the frame is 9.5 square centimeters. The film forming composition is dried to form a film layer. The film layer has a thickness of 50 microns.

Coral calcium powder (as a medicant) is combined with carboxymethylcellulose powder (as a fiber adhesive), modified food starch (as a soluble bulking agent), carrageenan (as adhesive), pullulan (as a polymer), calcium carbonate (as a non-soluble filler/bulking agent), sucralose (intense sweetener), talc (as flow/partitioning agent), and menthol (as a medicant) in a fluidized bed container. The resulting powder matrix includes 3.76% by weight of benzocaine powder, 5.2% by weight percent of carboxymethylcellusoe powder, 38.33% by weight of modified food starch, 5.0% by weight pullulan, 3.76% by weight menthol, 4% by weight carrageenan, 2.5% by weight talc, 0.45% by weight sucralose, 35% by weight calcium carbonate, and 2.0% by weight magnesium trisilicate.

The filler, fiber, and polymer components of the powder matrix are used to slow the dissolution of the medicant when the resulting medicant composition is placed in the oral mucosa of an individual.

The powder matrix is drawn from the fluidized bed container and is applied to the upper exposed surface of the film layer to a substantially uniform thickness of 80 microns. The powder matrix is atomized through a Nordson or similar static spray gun using compressed air. The powder matrix layer and film layer together comprise a medicant composition.

While the invention is described in terms of a specific embodiment, other embodiments could readily be adapted by one skilled in the art. Accordingly, the scope of the present invention is limited only by the following claims.

1. An edible film for the treatment of pharyngitis or cough comprising:
a film former; and
an active ingredient, wherein the edible film will dissolve when placed in the oral cavity thereby delivering the active ingredient to the oral cavity.

2. The edible film of claim 1 wherein the active ingredient comprises an ingredient selected from Table 1.

3. The edible film of claim 1 where in the active ingredient comprises menthol, benzocaine or both menthol and benzocaine.

4. The edible film of claim 1 comprising Water, N&A Cherry, Carrageen, Acsulfame Potassium, Sucralose, Lecithin, Benzocaine, Glycerin, Sodium Benzoate, Poly Sorbate 80 Menthol, Carboxymethyl Cellulose and one or more of Pectin, Gelatin, Maltodextrin, Modified Food Starch, TiO2, and Acacia Gum.

5. The edible film of claim 1 comprising:

| | |
|---|---|
| Water | about 0 to 25%; |
| N&A Cherry | about 0 to 25%; |
| Carrageen | about 0 to 10%; |
| Acsulfame Potassium | about 0 to .1%; |
| Sucralose | about 0 to 5%; |
| Lecithin | about 0 to 1%; |
| Benzocaine | about 0 to 12%; |
| Glycerin | about 0 to 10%; |
| Sodium Benzoate | about 0 to 2%; |
| Poly Sorbate 80 | about 0 to 0.5%; |
| Menthol | about 0 to 12%; |
| Carboxymethyl Cellulose | about 0 to 12%; and |
| Pectin | about 20 to 60%; |

wherein a portion of the Pectin may be replaced with one or more of the group consisting of Gelatin, Maltodextrin, Modified Food Starch, TiO2, and Acacia Gum.

6. The edible film of claim 1 comprising:

| | |
|---|---|
| Water | about 5 to 15%; |
| N&A Cherry | about 10 to 20%; |
| Carrageen | about 2 to 6%; |
| Acsulfame Potassium | about 0.2 to 0.6%; |
| Sucralose | about 1 to 3%; |
| Lecithin | about 0.2 to 0.6%; |
| Benzocaine | about 3 to 9%; |
| Glycerin | about 2 to 8%; |
| Sodium Benzoate | about 0.05 to 0.2%; |
| Poly Sorbate 80 | about 0.05 to 0.35%; |
| Menthol | about 3 to 9%; |
| Carboxymethyl Cellulose | about 3 to 9%; and |
| Pectin | about 35 to 50%; |

wherein a portion of the Pectin may be replaced with one or more of the group consisting of Gelatin, Maltodextrin, Modified Food Starch, TiO2, and Acacia Gum.

7. An edible film comprising:
a first layer; and
a second layer;
wherein the second layer comprising a dry coat layer having an active ingredient, and wherein the second layer is affixed to the first layer.

8. The edible film of claim 7 wherein the active ingredient comprises an ingredient selected from Table 1.

9. The edible film of claim 7 where in the active ingredient comprises menthol, benzocaine or both menthol and benzocaine.

10. The edible film of claim 7 comprising Water, N&A Cherry, Carrageen, Acsulfame Potassium, Sucralose, Lecithin, Benzocaine, Glycerin, Sodium Benzoate, Poly Sorbate 80 Menthol, Carboxymethyl Cellulose and one or more of Pectin, Gelatin, Maltodextrin, Modified Food Starch, TiO2, and Acacia Gum.

11. The edible film of claim 7 comprising:

| | |
|---|---|
| Water | about 0 to 25%; |
| N&A Cherry | about 0 to 25%; |
| Carrageen | about 0 to 10%; |
| Acsulfame Potassium | about 0 to .1%; |
| Sucralose | about 0 to 5%; |
| Lecithin | about 0 to 1%; |
| Benzocaine | about 0 to 12%; |
| Glycerin | about 0 to 10%; |
| Sodium Benzoate | about 0 to 2%; |
| Poly Sorbate 80 | about 0 to 0.5%; |
| Menthol | about 0 to 12%; |
| Carboxymethyl Cellulose | about 0 to 12%; and |
| Pectin | about 20 to 60%; |

wherein a portion of the Pectin may be replaced with one or more of the group consisting of Gelatin, Maltodextrin, Modified Food Starch, TiO2, and Acacia Gum.

12. The edible film of claim 7 comprising:

| | |
|---|---|
| Water | about 5 to 15%; |
| N&A Cherry | about 10 to 20%; |
| Carrageen | about 2 to 6%; |
| Acsulfame Potassium | about 0.2 to 0.6%; |
| Sucralose | about 1 to 3%; |
| Lecithin | about 0.2 to 0.6%; |
| Benzocaine | about 3 to 9%; |
| Glycerin | about 2 to 8%; |
| Sodium Benzoate | about 0.05 to 0.2%; |
| Poly Sorbate 80 | about 0.05 to 0.35%; |
| Menthol | about 3 to 9%; |
| Carboxymethyl Cellulose | about 3 to 9%; and |
| Pectin | about 35 to 50%; |

wherein a portion of the Pectin may be replaced with one or more of the group consisting of Gelatin, Maltodextrin, Modified Food Starch, TiO2, and Acacia Gum.

13. A method of ameliorating a cough or pharyngitis comprising:
placing an edible film comprising an active ingredient and a film former into the oral cavity wherein said film dissolves in the oral cavity to deliver the active ingredient to the oral cavity.

14. The method of claim 13 wherein the active ingredient comprises an ingredient selected from Table 1.

15. The method of claim 13 wherein the active ingredient comprises menthol, benzocaine or both menthol and benzocaine.

16. The method of claim 13 wherein the edible film comprises Water, N&A Cherry, Carrageen, Acsulfame Potassium, Sucralose, Lecithin, Benzocaine, Glycerin, Sodium Benzoate, Poly Sorbate 80 Menthol, Carboxymethyl Cellulose and one or more of Pectin, Gelatin, Maltodextrin, Modified Food Starch, TiO2, and Acacia Gum.

17. The method of claim 13 wherein the edible film comprises:

| | |
|---|---|
| Water | about 0 to 25%; |
| N&A Cherry | about 0 to 25%; |
| Carrageen | about 0 to 10%; |

Acsulfame Potassium about 0 to .1%;
Sucralose about 0 to 5%;
Lecithin about 0 to 1%;
Benzocaine about 0 to 12%;
Glycerin about 0 to 10%;
Sodium Benzoate about 0 to 2%;
Poly Sorbate 80 about 0 to 0.5%;
Menthol about 0 to 12%;
Carboxymethyl Cellulose about 0 to 12%; and
Pectin about 20 to 60%;
wherein a portion of the Pectin may be replaced with one or more of the group consisting of Gelatin, Maltodextrin, Modified Food Starch, TiO2, and Acacia Gum.

18. The method of claim 13 wherein the edible film comprises:

| | |
|---|---|
| Water | about 5 to 15%; |
| N&A Cherry | about 10 to 20%; |
| Carrageen | about 2 to 6%; |
| Acsulfame Potassium | about 0.2 to 0.6%; |
| Sucralose | about 1 to 3%; |
| Lecithin | about 0.2 to 0.6%; |
| Benzocaine | about 3 to 9%; |
| Glycerin | about 2 to 8%; |
| Sodium Benzoate | about 0.05 to 0.2%; |
| Poly Sorbate 80 | about 0.05 to 0.35%; |
| Menthol | about 3 to 9%; |
| Carboxymethyl Cellulose | about 3 to 9%; and |
| Pectin | about 35 to 50%; |

wherein a portion of the Pectin may be replaced with one or more of the group consisting of Gelatin, Maltodextrin, Modified Food Starch, TiO2, and Acacia Gum.

19. A method of ameliorating a cough or pharyngitis comprises:
placing an edible film into the oral cavity wherein said film dissolves in the oral cavity to deliver the active ingredient to the oral cavity and wherein the edible film comprises a first layer and a second layer, wherein the second layer comprises a dry coat layer comprising an active ingredient, and wherein the second layer is affixed to the first layer.

20. The method of claim 19 wherein the active ingredient comprises an ingredient
selected from Table 1.

21. The method of claim 19 wherein the active ingredient comprises menthol, benzocaine or both menthol and benzocaine.

22. The method of claim 19 wherein the edible film comprises Water, N&A Cherry, Carrageen, Acsulfame Potassium, Sucralose, Lecithin, Benzocaine, Glycerin, Sodium Benzoate, Poly Sorbate 80 Menthol, Carboxymethyl Cellulose and one or more of Pectin, Gelatin, Maltodextrin, Modified Food Starch, TiO2, and Acacia Gum.

23. The method of claim 19 wherein the edible film comprises:

| | |
|---|---|
| Water | about 0 to 25%; |
| N&A Cherry | about 0 to 25%; |
| Carrageen | about 0 to 10%; |
| Acsulfame Potassium | about 0 to .1%; |
| Sucralose | about 0 to 5%; |
| Lecithin | about 0 to 1%; |
| Benzocaine | about 0 to 12%; |
| Glycerin | about 0 to 10%; |
| Sodium Benzoate | about 0 to 2%; |
| Poly Sorbate 80 | about 0 to 0.5%; |
| Menthol | about 0 to 12%; |
| Carboxymethyl Cellulose | about 0 to 12%; and |
| Pectin | about 20 to 60%; |

wherein a portion of the Pectin may be replaced with one or more of the group consisting of Gelatin, Maltodextrin, Modified Food Starch, TiO2, and Acacia Gum.

24. The method of claim 19 wherein the edible film comprises:

| | |
|---|---|
| Water | about 5 to 15%; |
| N&A Cherry | about 10 to 20%; |
| Carrageen | about 2 to 6%; |
| Acsulfame Potassium | about 0.2 to 0.6%; |
| Sucralose | about 1 to 3%; |
| Lecithin | about 0.2 to 0.6%; |
| Benzocaine | about 3 to 9%; |
| Glycerin | about 2 to 8%; |
| Sodium Benzoate | about 0.05 to 0.2%; |
| Poly Sorbate 80 | about 0.05 to 0.35%; |
| Menthol | about 3 to 9%; |
| Carboxymethyl Cellulose | about 3 to 9%; and |
| Pectin | about 35 to 50%; |

wherein a portion of the Pectin may be replaced with one or more of the group consisting of Gelatin, Maltodextrin, Modified Food Starch, TiO2, and Acacia Gum.
